(19) United States
(12) Patent Application Publication
(10) Pub. No.: US 2002/0035260 A1
(43) Pub. Date: Mar. 21, 2002

(54) 4-aza-steroids

(75) Inventors: Chenchaiah C. Pamidi, Hamilton (CA); Qi Jia, Ajax (CA)

Correspondence Address: Nixon & Vanderhye P.C., 1100 North Glebe Road, 8th Floor, Arlington, VA, 22201, US

(73) Assignee: Novopharm Limited

(21) Appl. No.: 09/828,973

(22) Filed: Jul. 23, 2001

Related U.S. Application Data
(63) Continuation of application No. 09/423,386, filed on Jan. 28, 2000, now abandoned.
(60) Provisional application No. 60/045,810, filed on May 7, 1997.

(30) Foreign Application Priority Data
May 6, 1998 (CA) PCT/CA98/00438

(51) Int. Cl. C07J 73/00

(52) U.S. Cl. 546/77

(57) ABSTRACT

4-Aza-steroid compounds are provided, which have functional groups at one or more of positions 7, 11 and 15, such as hydroxyl or hydroxyl derivative groups. The compounds are active against 5-α-reductase giving indications of utility in combating prostate cancer. The compounds can be prepared by chemo-enzymatic synthesis from easily available 4-aza-steroids.

### FIELD OF THE INVENTION

This invention relates to 4-aza-steroids, processes for their preparation, and their pharmaceutical applications. More specifically, the invention relates to novel 4-aza-steroids useful both as pharmaceutical agents in the inhibition of the enzyme steroid 5-α-reductase, as intermediates in the preparation of other, novel, pharmaceutically active 4-aza-steroid compounds, and the novel, pharmaceutically active 4-aza-steroids preparable therefrom.

### BACKGROUND OF THE INVENTION AND PRIOR ART

The enzyme testosterone 5-α-reductase is known to cause reduction of testosterone in the body, to form dihydrotestosterone, DHT. DHT has been implicated in causing enlargement of the prostate, benign prostatic hyperplasia (BHP), leading to malignant conditions namely prostate cancer. Accordingly, it is desirable to inhibit the action of testosterone 5-α-reductase, and a number of 4-aza-steroids have been reported to be active in this respect. The best known of these is (5α, 17β)-(1,1-dimethyl-ethyl)-3-oxo-4-aza-androst-1-ene-17-carboxamide, commonly known as finasteride, of chemical structure:

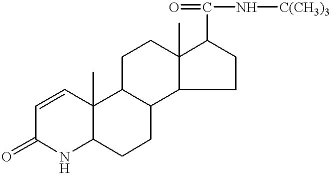

Finasteride has, since its original introduction, been reported to be less effective in treating BPH than originally expected (R. S. Rittmaster, N. Engl. J. Med., 1994, 330, 120-125). According to reports, there is room for further improvement in the level of residual circulating DHT (20-40%) in patients undergoing treatment with finasteride (G. J. Gormley et. al., J. Clin. Endocrinol. Metab., 1990, 70, 1136-1141).

It is now known that there are two isozymes of steroid reductase. The isozyme that principally interacts in skin tissue is conventionally designated as 5-α-reductase type I (present in rat ventral prostate), while the isozyme that interacts within the prostatic tissue is designated as 5-α-reductase type II (present in human prostate tissue and rat epididymus). It would be highly desirable to have one drug showing selectivity towards inhibiting 5-α-reductase type II isozyme, associated with benign prostatic hyperplasia and prostate cancer. It also would be highly desirable to have another drug showing selectivity towards 5-α-reductase type I isozyme associated with the scalp for use in treatment of male pattern baldness and hirsutism in females.

It is an object of the present invention to provide novel 4-aza-steroids having activity against testosterone 5-α-reductase.

### SUMMARY OF THE INVENTION

The present invention provides hydroxylated and other 4-aza-steroid compounds, said compounds having hydroxyl groups or other functional groups at one or both of the 7 and 15-positions. The novel compounds of the invention are active as inhibitors of testosterone 5-α-reductase type I and/or type II, and/or useful as chemical intermediates in preparing such active finasteride derivatives. They include both finasteride-type compounds and 1,2-dihydro-finasteride compounds.

The present invention also provides a novel microbiological process for preparing hydroxylated compounds of finasteride and 1,2-dihydro-finasteride, which comprises regio- and stereo-specific enzymatic oxidation reaction using a microorganism selected from the group consisting of Mortierella isabellina ATCC-42613, Bacillus megaterium ATCC-13368, Cunninghamella elegans ATCC-9244 and Cunninghamella elegans ATCC-9245, in a fermentation medium which supports the growth of the selected microorganism.

The present invention further provides a process of preparing novel finasteride and 1,2-dihydro-finasteride compounds having functional groups at one or more of positions 7-β, 11-α and 15-β, which comprises chemical reaction of the corresponding hydroxylated finasteride or 1,2-dihydro-finasteride compound with an appropriately chosen hydroxy-reactive chemical reagent capable of chemical conversion of the hydroxy group to the desired functional group.

Thus according to the present invention, there are provided novel finasteride derivatives corresponding to the general formula:

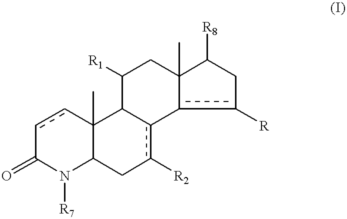

wherein solid bonds to substituents denote optional α or β stereo configurations and dotted lines in the nucleus denote optional unsaturation;

R and R₂ are independently selected from hydrogen; hydroxyl; halogen (F, Cl, Br, I); ester of formula —O—CO—R₃ where R₃ is hydrocarbyl selected from aliphatic (C₁-C₁₂), cycloalkyl (C₃-C₁₂), aromatic and aromatic-aliphatic such as benzyl, or heterocyclic (N, O or S), any of which are optionally unsaturated, optionally polybasic and optionally substituted with one or more substituents selected from alkyl, hydroxy, alkoxy, oxo, amino and halogen; sulphonic ester of formula —O—SO₂—R₄ where R₄ is hydrocarbyl aliphatic or aromatic of up to 12 carbon atoms; azide; amino; substituted amino of formula NR₃R₅ where R₃ is as defined above and R₅ is H or is independently selected from the radicals comprising R₃; and amino acyl of formula —NH—CO—R₆ or —NH—COOR₆ where R₆ is H or is independently selected from radicals comprising R₃;

R₁ is independently selected from the same group of radicals as R and R₂ but omitting hydroxy;

R₇ represent H or lower alkyl; with the proviso that R, R₁ and R₂ cannot all be hydrogen;

and R₈ is independently selected from hydrogen; hydroxyl; azide; oxo; halogen (F, Cl, Br, I); amino; substituted amino of formula NR₃R₅ where R₃ and R₅ are as defined above; amino acyl of formula —NH—CO—R₆ or —NH.CO.OR₆ where R₆ is H or is independently selected from the groups comprising R₃; —CO—R₉ or —CO—OR₉ or CO—NH—R₉ where R₉ is H or is independently selected from the groups comprising R₃.

### DESCRIPTION OF THE PREFERRED EMBODIMENTS

The preferred choice for group R₈ in formula I above is —CO—NH—R₉ where R₉ represents lower alkyl, especially t.butyl.

One preferred group of compounds according to the invention is that corresponding to the general formula:

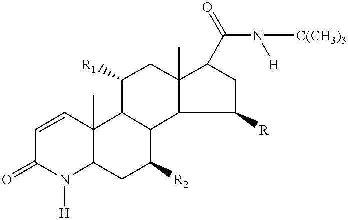

wherein at least one of the groups R, R₁ and R₂ represents a functional group chemically derivable from hydroxyl, and selected from halogen (F, Cl, Br, I); ester of formula —O—OC—R₃ where R₃ is aliphatic, cycloalkyl, aromatic, aromatic-aliphatic such as benzyl, or heterocyclic series (N, O or S atoms), any of which can be unsaturated and/or polybasic and/or conventionally substituted with substituents such as alkyl, hydroxy, alkoxy, oxo, amino, or halogen (F, Cl, Br, I); sulphonic ester of formula —O—O₂S—R₄ where R₄ is aliphatic or aromatic of 1-12 carbon atoms; azide-N₃; amino; substituted amino of formula —NR₃R₅ where R₃ is as shown above and R₅═R₃, H; amino acyl of formula —NH—CO—R₆ where R₆ ═R₃, OR₃.

One specific preferred compound according to the invention is 15-β-hydroxy-finasteride, of chemical structure:

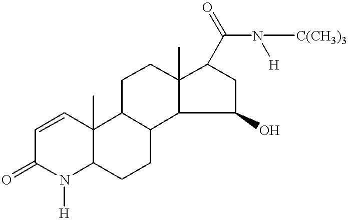

Conventional knowledge in organic chemistry can be utilized by those skilled in the art in converting the 15-β-hydroxy group of finasteride into its various novel 15-substituted compounds. Thus, 15-β-hydroxy-finasteride can be converted to various 15-substituted esters by the reaction of suitable acid halides or anhydrides in presence of esterifying agents such as trifluoroacetic anhydride (J. Org. Chem., 30, 927, 1965), dicyclohexylcarbodiimide (J. Org. Chem., 27, 4675, 1962), and acid catalysts such as sulphuric acid, hydrogen chloride, p-toluene sulphonic acid, methane sulphonic acid (Org. Synth. Coll. Vol. IV, 610, 1955). Esterification can also be performed on the hydroxyl group in the presence of suitable esterifying agents catalysed by a base. Suitable base catalysts are preferably tertiary amines such as pyridine, collidine triethylamine, 4-dimethylaminopyridine. Displacement of the halogen of any halogen ester with a suitable amine such as morpholine, piperidine, piperazine, N-methyl piperazine, dimethylamine, pyrrolidine, can form novel 15-substituted aminoesters of finasteride.

The 15-β-hydroxy-finasteride compound can be converted to 15-halo (F, Cl, Br, I) finasteride by reacting with appropriate halogenating reagents such as HCl, HBr, SOCl₂, PCl₃, PBr₃, PCl₅, POCl₃, an organic acid chloride or by reacting the 15-halo derivative (Cl, Br) with NaI. Those skilled in the art can use 15-halo- and/or 15-hydroxy-finasteride as an intermediate to synthesize various 15-substituted compounds, such as oxo, amino, amide, azido analogues and as well as Δ-14(15)-4-azasteroid, by known methods. Treatment of a 15-halo azasteroid with sodium azide to produce the 15-azido compound is an example of such chemical conversion. These azido compounds are themselves potent 5-alpha reductase enzyme inhibitors and serve as intermediates for synthesis of various 15-substituted amino azasteroids.

A second specific, preferred compound is 7-β-hydroxy-finasteride, of structure:

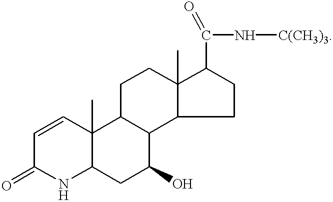

This is similarly convertible to halo, ester, azido, oxo, amino and amido derivatives, and to a Δ-7(8)-azasteroid.

Particularly preferred according to the present invention is the compound 7-α-chloro-finasteride, which can be prepared by reacting 7-β-hydroxy-finasteride with a chlorinating agent such as thionyl chloride in solution, followed by extraction and chromatographic purification. The 7-βchloro analog may be prepared in the same way. 7-α-chloro-finasteride has been found to have an activity against 5-α-reductase type II which is considerably higher than that of finasteride itself.

Similarly, the novel 7-α-azido-finasteride, prepared from 7-β-hydroxy-finasteride as shown in the following synthetic scheme, has also shown a very high specific inhibitory activity against 5-α-reductase type II.

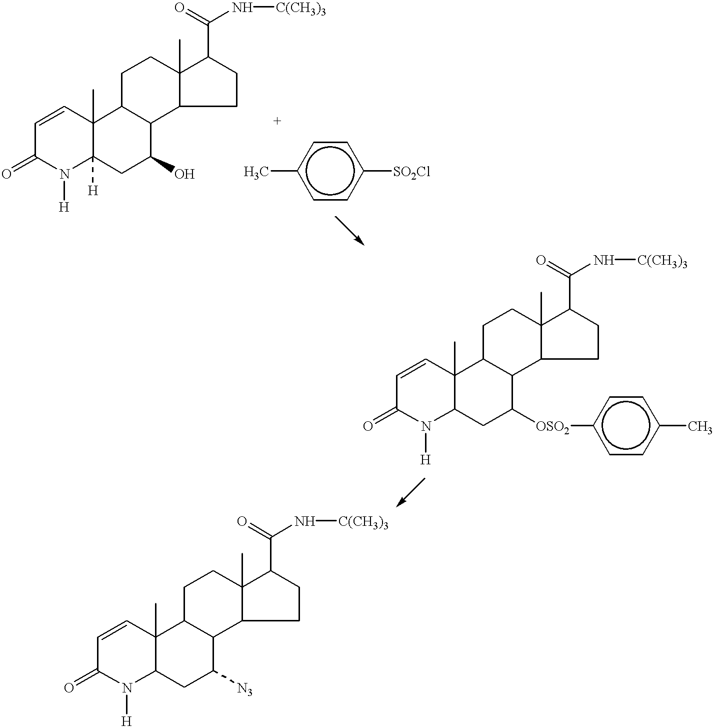

The process of the present invention, using as the microorganism Bacillus megaterium ATCC-13368, produces along with 15-β-hydroxy-finasteride, the compound 11-α-hydroxy-finasteride, of formula:

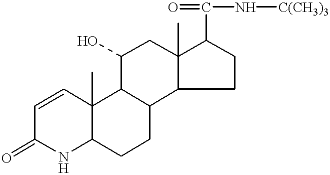

This compound can be similarly chemically converted at its 11-position to the corresponding halo, ester, amino, substituted amino, azido and Δ-9, 11 unsaturated derivatives which also form an aspect of the present invention.

One of the fungal microorganisms used in the process of the present invention, Mortierella isabellina ATCC-42613, is known to be capable of biochemical oxidation of organic compounds. It is commercially available. Suitable fermentation media for its growth are also known. However, its previous uses have been in oxidizing methyl groups —CH₃ to hydroxymethyl groups —CH₂OH in the side chains of organic compounds, such as oxidation of ethylbenzene to benzyl alcohol. Since finasteride possesses three terminal methyl groups on a side chain, it would have been expected that, if this microorganism had any action on finasteride at all, it would have been oxidation of one or more of these terminal methyl groups. Experimental work to date has shown that a small amount of such a product is indeed produced. It is most surprising and unexpected to find, in addition, that in its predominant reaction, Mortierella isabellina ATCC-42613 oxidizes C—H groups on the aza-steroid nucleus to C—OH.

Culturing the microorganism Mortierella isabellina ATCC-42613 in a fermentation broth in the presence of finasteride in fact leads to the production of a mixture of 4 different hydroxylated derivatives of finasteride, namely 11-α-hydroxy-finasteride, 15-β-hydroxy-finasteride (the major product) and 7-β-hydroxy-finasteride, of structural formulae given above, along with a small amount of ω-hydroxy finasteride.

Similarly, 1,2-dihydro-finasteride, a precursor of finasteride, as microbial biotransformation with Mortierella isabellina ATCC 42613 produced a mixture of different hydroxylated compounds of 1,2-dihydro-finasteride, namely 15-β-hydroxy-1,2-dihydro-finasteride and 7-β-hydroxy-1,2-dihydro-finasteride.

The microorganisms Cunninghamella elegans strains ATCC-9245 and ATCC-9244 used in the process of the present invention are more specific in their action. In a suitable growth medium, they convert finasteride in high yield to 15-β-hydroxy-finasteride, substantially selectively, without production of significant amounts of other finasteride derivatives. This microorganism is known and commercially available. Suitable fermentation media for its growth are also known. It has previously been proposed for use in dehydrogenation and oxidation of saturated aza-steroid compounds, see international patent application PCT/EP95/03992 (WO 96/12034) Poli et al.

The microorganism Bacillus megaterium ATCC-13368 used in the process of the present invention is also known and is commercially available, along with suitable growth media for its cultivation. It has previously been proposed for use in biochemical conversion of cyproterone acetate, another steroid, to 15-β-cyproterone acetate-see U.S. Pat. No. 4,337,311 Schering. In a suitable growth medium, Bacillus megaterium ATCC-13368 converts finasteride into the known 11-α-hydroxy-finasteride (see U.S. Pat. No. 5,215,894 Merck) and the novel 15-β-hydroxy-finasteride of the present invention, in an approximately 1:2 ratio.

The above described hydroxylation processes can also be carried out using the above-mentioned micro-organisms immobilized or using crude homogenates isolated from these organisms or purified enzymes isolated from these organisms or using them as biocatalysts. These experimental techniques are well known in the literature and can be carried out by those skilled in the art, see international patent application PCT/EP95/03992 (WO96/12034) Poli et al.

Pharmaceutical compositions, dosage forms and methods of administration, and dosage rates, for the compounds of the present invention are essentially similar to those for finasteride itself, and suitable such formulations and dosage rates can be determined by consulting the relevant published literature concerning finasteride.

The invention is further described, for illustrative purposes, in the following specific examples.

### EXAMPLE 1

Bioconversion of Finasteride using Mortierella isabellina, ATCC 42613

Nine 1 liter Erlenmeyer flasks each containing 200 ml of a nutrient solution of 4.0% dextrose, 0.5% yeast extract, 0.5% soytone, 0.5% sodium chloride, and 0.5% potassium phosphate dibasic, sterilized in an autoclave for 20 minutes at 121° C. were inoculated with a slope of culture of Mortierella isabellina ATCC 42613 kept on Malt Agar and kept shaking on an incubator shaker at 28° C. at 230 RPM for 3 days (68 hours). The combined fungal cells from all the flasks were filtered on a buchner funnel and washed with water. The resting cells were distributed among nine 1 liter Erlenmeyer flasks, each containing 150 ml of distilled water. A solution of finasteride (0.9 g) in 95% ethyl alcohol (9 ml) was distributed equally among the nine flasks and they were kept shaking at 28° C. at 230 RPM for 44 hours. The fungal biotransformation reaction was then worked up by filtering the fungal broth and extracting the medium with chloroform. The chloroform extract was dried over sodium sulfate and evaporated to dryness to afford the crude product which on TLC analysis showed the presence of four products and no starting material. Purification of crude product by column chromatography over silica by gradient elution with chloroform and methanol (90:10) afforded the desired novel fungal metabolites.

1) 15-β-hydroxy-finasteride (˜300 mg). ¹H-NMR (500 MHz; CDCl₃) δ: 0.96 s, 3H (CH₃ at 18); 0.99, s, 3H (CH₃ at 19); 1.33, s, 9H(t-butyl group); 3.32-3.35, m, 1H (CH-5; α-H); 4.25-4.28, m, 1H; 5.06, bs, 1H; 5.51, bs, 1H; 5.78-5.81, dd, 1H; 6.76-6.78, d, 1H.

MS(m/z): 389 (M+H); 388 (M^{+°}); 370 (M-H₂O); 355 (7.5%); 270.

2) 7-β-hydroxy-finasteride (˜200 mg). ¹H-NMR (500 MHz; CDCl₃; diagnostic signals) δ: 0.70 s, 3H (CH₃ at 18); 0.97, s, 3H (CH₃ at 19); 1.33, s, 9H(t-butyl group); 3.30-3.33, m, 1H (CH-5;α-H); 3.45-3.50, m, 1H; 5.07, bs, 1H; 5.66, bs, 1H; 5.79-5.81, dd, 1H; 6.75, d, 1H.

MS(m/z): 389 (M+H); 388 (m^{+°}); 370 (M-H₂O); 355; 270.

In addition to the above products, the purification yielded 20 mg. of ω-hydroxy-finasteride, the plasma metabolite and 70 mg. of 11-α-hydroxy-finasteride.

### EXAMPLE 2

Bioconversion of Finasteride using Cunninghamella elegans, ATCC 9245

Fourteen 1 liter Erlenmeyer flasks each containing 200 ml of a nutrient solution of 3% sabouraud dextrose broth, sterilized in an autoclave for 20 minutes at 121° C. were inoculated with a slope of culture of Cunninghamella eleqans ATCC 9245 kept on potato dextrose agar and kept shaking on an incubator shaker at 19-24° C. at 200 RPM for 71 hours. The combined fungal cells from all the flasks were filtered on a buchner funnel and washed with water. The resting cells were distributed among fourteen 1 liter Erlenmeyer flasks, each containing 150 ml of distilled water. A solution of finasteride (2.1 g) in 95% ethyl alcohol (14 ml) was distributed equally among the fourteen flasks and they were kept for shaking at 19-23° C. at 200 RPM for 73 hours. The fungal biotransformation reaction was then worked up by filtering the fungal broth and extracting the medium with chloroform. The chloroform extract was dried over sodium sulfate and evaporated to dryness to afford the crude product which on TLC analysis showed the presence of a single product. Purification of crude product by column chromatography over silica by gradient elution with chloroform and methanol (90:10) afforded 1.4 g of the desired 15-β-hydroxy-finasteride. The identity was confirmed by comparing on TLC with an authentic sample of 15-β-hydroxy-finasteride obtained from biotransformation of finasteride with Mortierella isabellina, ATCC 42613.

### EXAMPLE 3

Bioconversion of Finasteride using Cunninghamella elegans ATCC-9244

Nine 1 liter Erlenmeyer flasks each containing 200 ml of a nutrient solution of 3% sabouraud dextrose broth, sterilized in an autoclave for 20 minutes at 121° C. were inoculated with a slope of culture of Cunninghamella elegans ATCC 9244 kept on potato dextrose agar and kept shaking on an incubator shaker at 28° C. at 200 RPM for 90 hours. The combined fungal cells from all the flasks were filtered on a buchner funnel and washed with water. The resting cells were distributed among nine 1 liter Erlenmeyer flasks, each containing 150 ml of distilled water. A solution of finasteride (1.35 g) in 95% ethyl alcohol (9 ml) was distributed equally among the nine flasks and they were kept shaking at 28° C. at 200 RPM for 74 hours. The fungal biotransformation reaction was then worked up by filtering the fungal broth and extracting the medium with chloroform. The chloroform extract was dried over sodium sulfate and evaporated to dryness to afford the crude product by column chromatography over silica by gradient elution with chloroform and methanol (90:10) afforded 1.1 g of the desired 15-β-hydroxy-finasteride. The identity was confirmed by comparing on TLC with an authentic sample of 15-β-hydroxy-finasteride obtained from biotransformation of finasteride with Mortierella isabellina, ATCC 42613.

### EXAMPLE 4

Bioconversion of Finasteride using Bacillus Megaterium, ATCC 13368

Nine 1 liter Erlenmeyer flasks each containing 200 ml of a nutrient solution (pH adjusted to 7.24 with 1N. sodium hydroxide) of 4% yeast extract and 1.5% soytone, sterilized in an autoclave for 20 minutes at 121° C. were inoculated with a slope of culture of Bacillus megaterium, ATCC 13368 kept on nutrient agar and kept shaking on an incubator shaker at 28° C. at 200 RPM for 72 hours. A solution of finasteride (1.35 g) in 95% ethyl alcohol (9 ml) was distributed equally among the nine Erlenmeyer flasks containing the bacterial suspension and they were kept shaking at 28° C. at 200 RPM for 24.5 hours. The bacterial biotransformation reaction was then worked up by combining the bacterial broth and extracting it with chloroform. The chloroform extract was dried over sodium sulfate and evaporated to dryness to afford a crude product which on comparative TLC analysis showed the presence of two products, 11-α-hydroxy and 15-β-hydroxy compounds of finasteride. Purification of crude product by column chromatography over silica by gradient elution with chloroform and methanol (95:5) afforded 0.49 g of 11-α-hydroxy-finasteride and 0.85 g of 15-β-hydroxy-finasteride. The identity was confirmed by comparing on TLC with the authentic samples of 11-α-hydroxy-finasteride and 15-β-hydroxy-finasteride, obtained from biotransformation of finasteride with Mortierella isabellina ATCC 42613.

### EXAMPLE 5

Preparation of 15-β-acetoxy-finasteride

15-β-hydroxy-finasteride (150 mg), taken in tetrahydrofuran (7 ml) and chloroform (3 ml), was allowed to react with acetyl chloride (82 μl) and pyridine (0.28 ml) at room temperature overnight. The reaction mixture was mixed with water and extracted with chloroform. Evaporation of the dried solvent followed by chromatographic purification with chloroform and methanol (95:5) afforded 15-β-acetoxy-finasteride (130 mg) as a colourless solid.

¹H-NMR (500 MHz; CDCl₃; diagnostic signals) δ: 0.91 s, 3H (CH₃ at 18); 1.00, s, 3H (CH₃ at 19); 1.33, s, 9H (t-butyl group); 2.00, s, 3H(—OCOCH₃); 3.30-3.34, t. 1H; 5.04, s, 1H; 5.09-5.12, m, 1H; 5.51, s, 1H; 5.79-5.81, dd, 1H; 6.75-6.77, d, 1H.

MS(m/z): 430 (M⁺); 370 (M-CH₃COOH); 270; 110.

### EXAMPLE 6

Preparation of 7-β-acetoxy-finasteride

7-β-hydroxy-finasteride (150 mg), taken in chloroform (5 ml), was allowed to react with acetyl chloride (82 μl) with pyridine (0.281 ml) at room temperature overnight. The reaction mixture was mixed with water and extracted with chloroform, washed with 1N HCl, water, saturated sodium bicarbonate solution and dried over sodium sulfate. Evaporation of the dried solvent followed by chromatographic purification with chloroform and methanol (97:3) and crystallization from chloroform and hexane afforded a colourless solid (61 mg).

¹H-NMR (500 MHz; CDCl₃; diagnostic signals)δ: 0.71 s, 3H (CH₃ at 18); 0.99, s, 3H (CH₃ at 19); 1.33, s, 9H (t-butyl group); 2.01, s, 3H (—OCOCH₃); 3.35-3.38, m, 1H (C-5; α-H); 4.59-4.65, m, 1H (7-α-H); 5.06, s, 1H (NH); 5.59, s, 1H (NH); 5.81-5.83, d, 1H (CH at 2); 6.73-6.75, d, 1H (CH at 1).

MS (m/z): 430 (M⁺); 370 m-CH₃COOH)⁺

### EXAMPLE 7

Preparation of 7-α-chloro-finasteride

A mixture of 7-β-hydroxy-finasteride (208 mg), benzene (15 ml) and thionyl chloride (0.4) was stirred at room temperature overnight. The reaction mixture was mixed with water, the pH was adjusted to 10 and extracted with chloroform, washed with 1N HCl, water, saturated sodium bicarbonate solution and dried over sodium sulfate. Evaporation of the dried solvent followed by chromatographic purification of the resultant crude product with chloroform and methanol (95:5) and crystallization from chloroform and hexane afforded 7-α-chloro-finasteride as a colorless solid (47 mg).

¹H-NMR (500 MHz; CDCl₃; diagnostic signals)δ; 0.69, s, 3H (CH₃ at 18); 0.97, s, 3H (CH₃ at 19); 1.33, s, 9H (t-butyl group); 3.95-3.98, m, 1H (CH-5; α-H; 0.63 ppm deshielded suggests Cl is in 7-α-position); 4.31, d, 1H (7-β-H); 5.06, s, 1H; 5.60, s, 1H; 5.81-5.83, dd, 1H; 6.75-6.67, d, 1H.

MS (m/z): 406 (M⁺); 371 (M-Cl); 270-110

### EXAMPLE 8

Preparation of 7-β-tosyloxy-finasteride

To a solution of 7-β-hydroxy-finasteride (200 mg) in pyridine (5 ml) at 0-5° C. was added p-toluene sulphonyl chloride (215 mg). The resultant mixture was kept in the refrigerator. TLC analysis suggested that there was still unreacted starting material. Another 220 mg of p-toluene sulphonyl chloride was added and kept in the refrigerator. Reaction mixture was poured into ice cold water, pH was adjusted to 3 with 5N HCl and it was extracted with chloroform, washed with water dried over sodium sulfate. Evaporation of the solvent followed by chromatographic purification of the crude product with chloroform and methanol (92:8) and crystallization afforded 7-β-tosyloxy-finasteride as a colorless solid (90 mg).

¹H-NMR (500 MHz; CDCl₃; diagnostic signals)δ: 0.66, s, 3H (CH₃ at 18); 0.93, s, 3H (CH₃ at 19); 1.31, s, 9H (t-butyl group); 2.44, s, 3H; 3.24-3.27, dd, 1H; 4.48-4,54, m 1H; 5.07, s, 1H; 5.18, s, 1H; 5.78-5.80, d, 1H; 6.70-6.72, d, 1H; 7.31-7.33, d, 2H; 7.75-7.77, d, 2H.

MS (m/z): 543 (M+H)⁺

### EXAMPLE 9

Preparation of 7-α-azido-finasteride

A mixture of 7-β-tosyloxy-finasteride (50 mg), sodium azide (55 mg) in DMF (3 ml) was stirred at RT overnight. TLC analysis suggested that there was still unreacted starting material. Another 10 mg of sodium azide was added and kept stirring overnight. Reaction mixture was poured into water, extracted with ether, washed with water, dried over magnesium sulfate and evaporation of the solvent afforded 7-α-azido-finasteride, a colorless solid.

¹H-NMR (500 MHz; CDCl₃; diagnostic signals)δ: 0.68, s, 3H (CH₃ at 18); 0.95, s, 3H (CH₃ at 19); 1.33, s, 9H (t-butyl) 3.71-3.75, dd, 1H (7-β-H; equatorial); 3.80, s, 1H (CH-5; α-H; 0.5 ppm deshielded suggests N₃ is in 7-α-position); 5.05, s, 1H; 5.80-5.82, d, 1H; 6.72-6.74, d, 1H.

MS (m/z): 414 (M+H)⁺

### EXAMPLE 10

Preparation of 14,15-dehydro-finasteride

To a mixture of 15-β-hydroxy-finasteride (206 mg) in benzene (10 ml), was added a solution of thionyl chloride (1.0 ml) in benzene (5 ml), and the resultant mixture was stirred at room temperature overnight. TLC indicated that the starting material has disappeared. The reaction mixture was added with water, pH was adjusted to 10, extracted with chloroform, the solvent extract was washed with 1N HCl and saturated sodium bicarbonate solution and dried over sodium sulfate. The resultant crude product, after purification by column chromatography (chloroform: MeOH; 95:5) and crystallization from chloroform and hexane, afforded a colorless solid (108 mg), expected to be the intermediate, 15-chloro-finasteride. A mixture of the intermediate (50 mg) and sodium hydroxide (8 mg) were stirred in methanol (3 ml) at room temperature overnight. Water (3 ml) was added to the reaction mixture and was extracted with chloroform (2×10 ml) after the pH was adjusted to 3 with 1N HCl. The organic extract was washed with saturated sodium bicarbonate and dried over sodium sulfate. Evaporation of the solvent followed by chromatographic purification of the crude product with chloroform and methanol (95:5) and crystallization from ether afforded 14,15-dehydro-finasteride as a colorless solid (27 mg).

¹H-NMR (500 MHz; CDCl₃; diagnostic signals)δ: 0.94, 0.97, 2 siglets, 6H (2 CH₃ at 18 and 19); 1.35, s, 9H (t-butyl); 3.28-3.31, m, 1H (CH-5); 5.11, s, 1H; 5.17, s, 1H; 5.24, s, 1H; 5.79-5.82, dd, 1H; 6.73-6.75, d, 1H.

MS (m/z: 370 (M⁺).

### EXAMPLE 11

Preparation of 1,2-dihydro-15β-hydroxy-finasteride

15-β-hydroxy-finasteride (70 mg) was hydrogenated over 10% Pd/C (7 mg) in absolute ethanol (10 ml) at room temperature under atmospheric pressure with stirring for five days. The reaction mixture was filtered, solids washed with ethanol, the combined alcohol extracts were evaporated off to give a residue. Crystallization of the resultant crude product from chloroform and hexane afforded 1,2-dihydro-15-β-hydroxy-finasteride as a colorless solid (41 mg).

¹H-NMR (500 MHz; CDCl₃; diagnostic signals)δ: 0.92, s, 3H (CH₃ at 18); 0.95, s, 3H (CH₃ at 19); 1.33, s, 9H (t-butyl group); 3.04-3.07, dd, 1H (CH-5; α-H); 4.27, s, 1H (15-α-H); 5.06, s, 1H, 5.65, s, 1H.

MS (m/z): 391 (M+H)⁺

### EXAMPLE 12

Preparation of 1,2-dihydro-7-β-hydroxy-finasteride

7-β-hydroxy-finasteride (50 mg) was hydrogenated over 5 10% Pd/C (7 mg) in absolute ethanol (10 ml) at room temperature under atmospheric pressure with stirring for five days. The reaction mixture was filtered, solids were washed with ethanol. The combined ethanol extract was concentrated to afford 1,2-dihydro-7-β-hydroxy-finasteride as a colorless solid (22 mg).

¹H-NMR (500 MHz; CDCl₃; diagnostic signals)δ: 0.70, s, 3H (CH₃ at 18); 0.92, s, 3H (CH₃ at 19); 1.33, s, 9H (t-butyl); 3.05-3.08, dd, 1H (CH-5; α-H); 3.42-3.45, m, 1H (7-α-H); 3.69, s, 1H, 5.07, s, 1H; 5.50, s, 1H.

MS (m/z): 391 (M+H)⁺

### EXAMPLE 13

Bioconversion of 1,2-dihydro-finasteride using Mortierella isabellina ATCC 42613

Following the procedure as described in Example 1, the microbial biotransformation was carried out on 1,2-dihydro-finasteride using Mortierella isabellina, ATCC 42613. Thus, fungal broth, obtained from biotransformation reaction of 1,2-dihydro-finasteride (3.0 g) for 69 hours, was extracted with chloroform. The chloroform extract was dried over sodium sulfate and evaporated to dryness to afford a crude product (4.29 g) which on purification by column chromatography over silica by gradient elution with chloroform, and methanol (95:5) afforded the desired novel fungal metabolites.

1) 1,2-Dihydro-15-β-hydroxy-finasteride (1.2 g). NMR and M/S are identical to that of Example 11.

2) 1,2-Dihydro-7-β-hydroxy-finasteride 1.2 g). NMR and M/S are identical to that of Example 12.

### EXAMPLE 14

Preparation of 1,2-dihydro-7-α-chloro-finasteride

A mixture of 1,2-dihydro-7-β-hydroxy-finasteride (50 mg), benzene (5 ml) and thionyl chloride (0.5 ml) was stirred at room temperature for five days. Reaction mixture was mixed with chloroform (40 ml) and water (20 ml) and stirred for 10 minutes. The aqueous extract was washed with water, saturated sodium bicarbonate solution and dried over sodium sulfate.

Evaporation of the dried solvent followed by chromatographic purification of the resultant crude product with chloroform and crystallization from chloroform and hexane afforded 1,2-dihydro-7-α-chloro-finasteride as a colorless solid (30 mg).

¹H-NMR (500 MHZ; CDCl₃; diagnostic signals)δ: 0.68, s, 3H (CH₃ at 18); 0.91, s, 3H (CH₃ at 19); 1.33, s, 9H (t-butyl group); 3.67-3.70, t, 1H (CH-5; α-H; 0.63 ppm deshielded suggests Cl is in a 7-α-position); 4.31, d, 1H (7-β-H); 5.04, s, 1H; 5.46, s, 1H.

MS (m/z): 408 (M⁺).

### EXAMPLE 15

Biochemical Assays

Biochemical Assays were carried out to determine the inhibitory activities of various compounds of the previous examples on 5-α-reductase I enzyme isolated from male rate prostate and 5-α-reductase II enzyme isolated from rat epididymus and human prostate. These procedures were carried out following published literature procedures (H. Takami et al., J. Med. Chem., 39, pp 5047-5052; Tehming Liang, Margaret A. Cascieri et al., Endocrinology, 117, pp 571-579). Brief descriptions are as follows:

Rat 5-α-reductase I enzyme assay. Prostates, removed from 16 young male Sprague dawley rats (each weighing about 300-400 g), were minced and homogenized at 0-4° C. in 3 tissue volumes of buffer (0.32 M sucrose, 1 mM dithiothreitol, and 20 mM phosphate buffer, pH 6.5) using a polytron homogenizer. The homogenate was centrifuged at 4° C. at 140,000 g for 1 hour. The resultant pellet, after washing with the homogenizing buffer was suspended in the same buffer and stored at −70° C. The assay was carried out in a final volume of 0.5 ml containing 20 mM phosphate buffer (pH 6.5), 1 mM dithiothreitol, 150 μM NADPH, 2 μM ¹⁴C testosterone and the enzyme concentration (500 μg-1 mg) For conducting the inhibitory studies on a 5-α-reductase I, finasteride and other test compounds were added in 10 μl of ethanol to a concentration 10⁻⁹ to 10⁻⁵ with five to six points including control using duplicate for each point to the above reaction mixture. The incubations were done for 20 minutes at 37° C. The reactions were stopped by adding 2.0 ml of ethyl acetate containing testosterone, 5-α-dihydrotestosterone, and androstenedione (10 μg each). After centrifugation at 1000 g for 5 minutes, the upper ethyl acetate extract was transferred to a tube and then evaporated under nitrogen to dryness. The compounds were taken up in 50 μl of ethyl acetate and chromatographed on Whatman LK5DF silica GF TLC plates using ethyl acetate-cyclohexane (1:1). The respective TLC spots corresponding to testosterone and dihydrotestosterone (Rf value same as that of androstenedione) were scraped from the plate and taken in respective scintillation vials. They were counted in the Beckman scintillation counter model No. LS 6500 with counting efficiency of 95% for ¹⁴C carbon. Finasteride was used as a known standard during all screening. The range of IC₅₀ values for different test compounds obtained from different experiments is shown in Table 1 under the column, Rat Prostate Enzyme I IC₅₀.

Rat 5-α-reductase II enzyme assay: Epididymus, taken out during the isolation of the rat prostates during rat enzyme I assay, was stored at −70° C. Isolation of the enzyme and the assay were carried out following the procedure described above, except the reaction buffer used was 40 mM Tris-citrate, pH 4.5. The range of IC₅₀ values for different test compounds obtained from different experiments is shown in Table 1 under the column, Rat Epididymus Enzyme II IC₅₀.

Human 5-α-reductase II enzyme assay: Specimens of human prostates were quickly frozen in dry ice after collection and kept at −70° C. before isolation of the enzyme. Isolation of the enzyme and the assay were carried out following a similar procedure as for the isolation of rat 5-α-reductase II enzyme with some modifications. During the isolation of the enzyme, 50 μM NADPH was added to the homogenizing buffer as a stabilizer. The enzyme was stored in the homogenizing buffer containing 20% glycerol. The enzyme reaction buffer used as 40 mM Tris-citrate buffer, pH 5.0. The range of IC₅₀ values for different test compounds obtained from different experiments is shown in Table 1 under the column, Human Prostate Enzyme II IC₅₀.

TABLE 1
BIOCHEMICAL ASSAYS

| | | | Rat | Human |
|---|---|---|---|---|
| Compound | | Rat Prostate | Epididymus | Prostate |
| of | | Enzyme I | Enzyme II | Enzyme II |
| Example | Compound | IC₅₀ | IC₅₀ | IC₅₀ |
| | Finasteride | 13-30 | ˜2.9-11.8 | 3.3-7 |
| | | nM | nM | nM |
| | Finasteride I | 11.0-38 | 5.1-14.8 | 2.7-10.6 |
| | given as a blind | nM | nM | nM |
| | compound to test | | | |
| | the assay results | | | |
| 1,2,3,4 | 15-β-hydroxy- | 805-853 | 640 nM- | 50-59 |
| | finasteride | nM | 1.01 nM | nM |
| 1 | 7-β-hydroxy- | 811-1800 | 106 | 62-64 |
| | finasteride | nM | nM | nM |
| 5 | 15-β-Acetoxy- | 4.2-5.7 | 1.6-2.7 | 2.01 |
| | finasteride | μm | μM | μM |
| 6 | 7-β-Acetoxy- | 385-697 | 135-357 | 175-362 |
| | finasteride | nM | nM | nM |
| 7 | 7-α-Chloro- | 306-350 | 1.92-3.1 | 0.9-3.6 |
| | finasteride | nM | nM | nM |
| 8 | 7-β-Tosyloxy- | 1.5-4.3 | incomplete | 213-570 |
| | finasteride | μM | | nM |
| 9 | 7-α-Azido- | 577-1.1 | incomplete | 19-32 |
| | finasteride | μM | | nM |
| 10 | 14,15-Dehydro- | 50-67 | 45 | 39-49.6 |
| | finasteride | nM | nM | nM |
| 11,13 | 1,2-Dihydro-15- | 351-468 | incom- | incom- |
| | β-hydroxy- | nM | plete | plete |
| | finasteride | | | |
| 12,13 | 1,2-Dihydro-7-β- | 453-492 | 567-891 | 536-755 |
| | hydroxy- | nM | nM | nM |
| | finasteride | | | |
| 14 | 1,2-Dihydro-7-α- | 53-99 | 2.9-8.6 | 11.5-44 |
| | chloro- | nM | nM | nM |
| | finasteride | | | |

1. 4-Aza-steroid compounds corresponding to the general formula: 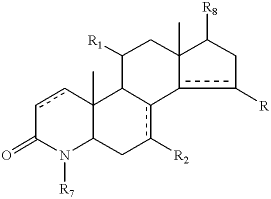
wherein solid bonds to substituents denote optional α or β stereoconfigurations and dotted lines in the nucleus denote optional unsaturation;
R and R₂ are independently selected from hydrogen; hydroxyl; halogen (F, Cl, Br, I); ester of formula —O—CO—R₃ where R₃ is hydrocarbyl selected from aliphatic (C₁-C₁₂), cycloalkyl (C₃-C₁₂), aromatic and aromatic-aliphatic such as benzyl, or heterocyclic (N, O or S), any of which are optionally unsaturated, optionally polybasic and optionally substituted with one or more substituents selected from alkyl, hydroxy, alkoxy, oxo, amino and halogen; sulphonic ester of formula —O—SO₂—R₄ where R₄ is hydrocarbyl aliphatic or aromatic of up to 12 carbon atoms; azide; amino; substituted amino of formula NR₃R₅ where R₃ is as defined above and R₅ is H or is independently selected from the radicals comprising R₃; and amino acyl of formula —NH—CO—R₆ or —NH—COOR₆ where R₆ is H or is independently selected from radicals comprising R₃;
R₁ is independently selected from the same group of radicals as R and R₂ but omitting hydroxyl;
R₇ represent H or lower alkyl;

4. Compounds according to any preceding claim wherein R₇ is hydrogen.

5. A compound according to claim 1 which is 1,2-dihydro-7-α-chloro-finasteride.

6. A compound according to claim 1 which is 1,2-dihydro-7-β-hydroxy-finasteride.

7. A compound according to claim 1 which is 14,15-dehydro-finasteride.

8. A compound according to claim 1 which is 15-β-hydroxy-finasteride.

9. A compound according to claim 1 which is 7-β-hydroxy-finasteride.

10. A compound according to claim 1 which is 7-α-chloro-finasteride.

11. A compound according to claim 1 which is 7-β-tosyloxy-finasteride.

12. A compound according to claim 1 which is 7-α-azido-finasteride.

13. Pharmaceutical composition useful in inhibiting 5-α-reductases and comprising an effective amount of a 4-aza-steroid compound as defined in any of claims 1-12.

14. A process of preparing hydroxylated derivatives of finasteride, which comprises biochemically hydroxylating finasteride by the action of a microorganism selected from the group consisting of Mortierella isabellina ATCC-42613, Cunninghamella elegans ATCC-9244 and Cunninghamella elegans ATCC-9245, in a fermentation medium which supports the growth of the selected microorganism.

15. A process for preparing finasteride derivatives having functional groups at one or more of position 7-β, 11-α and 15-β, which comprises preparation of the corresponding hydroxylated finasteride by the process of claim 14, and chemically reacting the hydroxylated finasteride with an appropriately chose hydroxy-reactive chemical reagent capable of chemical conversion of the hydroxy group to the desired functional groups.

16. A process for preparing hydroxylated derivatives of 1,2-dihydro-finasteride, which comprises biochemically hydroxylating 1,2-dihydro-finasteride by the action of the microorganism Mortierella isabellina ATCC 42613 in a fermentation medium which supports the growth of the microorganism.

17. A process for preparing 1,2-dihydro-finasteride compound having functional groups at one or more of positions 7-β, 11-α and 15-β, which comprises preparation of the corresponding hydroxylated1,2-dihydro-finasteride compounds by the process of claim 16, and chemically reacting the hydroxylated 1,2-dihydro-finasteride so prepared with an appropriately chosen hydroxy-reactive chemical reagent capable of chemical conversion of the hydroxy group to the desired functional group.
